(19) United States
(12) Patent Application Publication
(10) Pub. No.: US 2021/0315280 A1
(43) Pub. Date: Oct. 14, 2021

(54) DETERMINING A DIAGNOSTIC ASSOCIATED WITH AN ELECTRONIC SMOKING ARTICLE

(71) Applicant: Fontem Holdings 1 B.V., AMSTERDAM (NL)

(72) Inventors: Ramon Alarcon, Los Gatos, CA (US); Stellios Leventis, San Francisco, CA (US); Michael Starman, Los Gatos, CA (US)

(21) Appl. No.: 17/272,109

(22) PCT Filed: Aug. 28, 2019
(86) PCT No.: PCT/IB2019/057262

§ 371 Date: Feb. 26, 2021

Related U.S. Application Data
(60) Provisional application No. 62/723,738, filed on Aug. 28, 2018.

(51) Int. Cl. A24F 40/53; A24F 40/65; A24F 40/95; A24F 40/51; G06Q 30/02; G06Q 30/00; G06F 8/65

(52) U.S. Cl. CPC A24F 40/53; A24F 40/65; A24F 40/95; G06Q 10/20; G06Q 30/0217; G06Q 30/016; G06F 8/65; A24F 40/51

(57) ABSTRACT

Systems, methods, and devices for determining a diagnostic associated with an electronic smoking article (100, 108) are discussed herein. A connection with an electronic smoking article (100, 108) can be created with a computer (250). Data can be transferred between a computer (250) and the electronic smoking article (100, 108), upon connection with the electronic smoking article (100, 108). In some embodiments, a diagnostic associated with the electronic smoking article (100, 108) can be determined based upon the transferred data.

### BACKGROUND

### a. Field of the Disclosure

This disclosure relates to determining a diagnostic associated with an electronic smoking article.

### b. Background Art

Electronic cigarettes are a popular alternative to traditional smoking articles that bum tobacco products to generate smoke for inhalation. Unlike traditional tobacco-based smoking articles, electronic cigarettes generate an aerosol-based vapor for inhalation, which can generally emulate smoke of traditional tobacco-based smoking articles. The aerosol-based vapor can generally be created through heating of a liquid that contains additives, for example, nicotine. The heater can be powered by a power source, such as a battery. In some instances the battery can be rechargeable.

### SUMMARY

In various embodiments, a connection with an electronic smoking article can be created with a computer. Data can be transferred between a computer and the electronic smoking article, upon connection with the electronic smoking article. In some embodiments, a diagnostic associated with the electronic smoking article can be determined based upon the transferred data.

In various embodiments, a data connection between an electronic smoking article and a computer can be established. Data can be received from the electronic smoking article. An operation can be performed on the transferred data to determine a diagnostic related to the electronic smoking article. The diagnostic can be associated with an error in operation of the electronic smoking article.

In various embodiments, an electronic smoking article can be recognized upon connection of the electronic smoking article to a computer. A charge can be provided to the electronic smoking article via the connection. An amount of charge provided to the electronic smoking article can be determined via the connection. In some embodiments, a usage associated with the electronic smoking article can be determined based upon the amount of charge provided to the electronic smoking article.

### BRIEF DESCRIPTION OF THE DRAWINGS

FIG. 1A is an isometric front, side, and top view of a pack (or case or carrier) for electronic cigarettes, in accordance with embodiments of the present disclosure.

FIG. 1B is an isometric front, side, and top view of an alternative pack for electronic cigarettes, in accordance with embodiments of the present disclosure, shown with its lid open and holding three electronic cigarettes.

FIG. 1C is an isometric bottom and side view of the pack for electronic cigarettes shown in FIG. 1B.

FIG. 2 is a schematic overview of a system incorporating a pack for electronic cigarettes, an electronic cigarette, a computer, and an external power source, in accordance with embodiments of the present disclosure.

FIG. 3 is a method flow diagram that depicts a method for determining a diagnostic associated with an electronic smoking article.

FIG. 4 is a diagram of a computing device for determining a diagnostic associated with an electronic smoking article.

### DETAILED DESCRIPTION

As used herein, an “electronic smoking article” can include, for example, an electronic cigarette and/or a pack (or case or carrier) configured for storing one or more of a plurality of electronic cigarettes. However, “electronic smoking articles” can include other devices associated with electronic cigarettes not mentioned herein. Electronic cigarettes can include various componentry to enable a substrate (e.g., a liquid, paste, solid, or other medium) to be vaporized by the electronic cigarette, such that the resulting vapor can be inhaled by a user. The various components can include, for example, a heating element that can be energized by a power source (e.g., battery). In some embodiments, the substrate can be drawn to the heating element via a wick through capillary action or by other transfer means. Upon heating of the substrate, a vapor is generated that can be inhaled by the user.

Electronic smoking articles can include software, firmware, and/or hardware for executing various instructions to control different components in the electronic smoking articles. For example, instructions can provide for control of social networking functionality associated with a pack configured for storing one or more of a plurality of individual electronic cigarettes. Alternatively, and/or in addition, the instructions can provide for control over the function of (a) the heater associated with each individual electronic cigarette, (b) the battery associated with the pack and/or each individual electronic cigarette, (c) the sensors associated with either the pack or each individual electronic cigarette, and/or (d) other components associated with the electronic smoking articles.

Although the instructions can provide for control over different components in the electronic smoking articles, errors can exist in the instructions, which can cause problems with the control or function of the different components. Alternatively, the instructions can become outdated, such that the electronic smoking article does not function properly or as desired. For instance, it may be difficult to establish communications with the electronic smoking article under certain circumstances.

The methods according to the present disclosure can include determining a diagnostic associated with an electronic smoking article and/or performing an action with respect to the determined diagnostic. For instance, devices and methods according to the present disclosure can provide updated instructions (e.g., firmware or software) to the electronic smoking article; provide for troubleshooting related to the usage of the electronic smoking article; and/or provide for a determination of various metrics associated with the electronic smoking article.

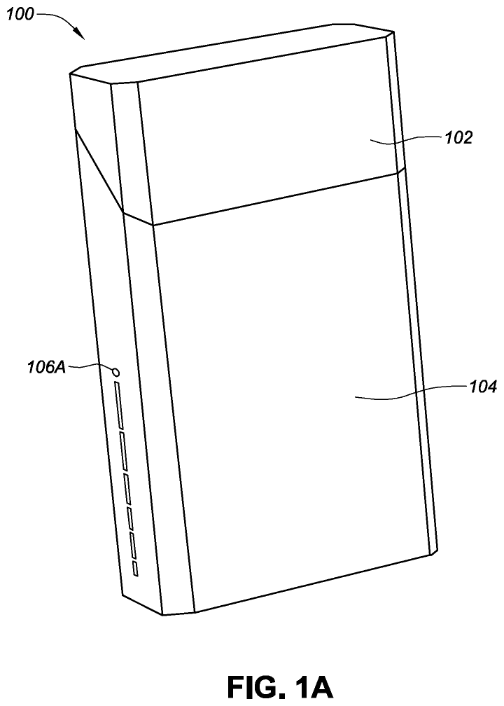

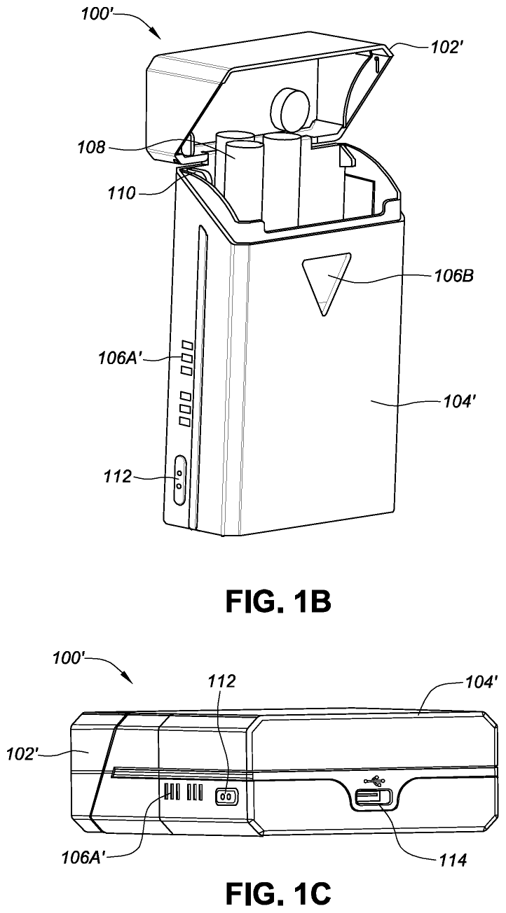

Referring now to the drawings wherein like reference numerals are used to identify identical components in the various views, FIG. 1A is an isometric front, side, and top view of a pack 100 for electronic cigarettes, in accordance with embodiments of the present disclosure. In some examples, the pack 100 can be configured for storing a plurality of electronic cigarettes. The pack 100 can include a lid 102 and a main body 104, which can include a living or mechanical hinge to connect the lid 102 to the main body 104, allowing for movement of the lid 102 with respect to the main body 104. The pack 100 may also include one or more indication lights 106A in some embodiments for providing notifications to a user.

FIG. 1B is an isometric front, side, and top view of an alternative pack 100′ for electronic cigarettes, in accordance with embodiments of the present disclosure, shown with its lid 102′ open and holding three electronic cigarettes 108. As discussed herein, an “electronic smoking article” can include a pack (e.g., 100 or 100′) configured for storing a plurality of individual electronic cigarettes 108. In addition, an “electronic smoking article” can include one or more of the plurality of individual electronic cigarettes 108. The pack 100′ can include the lid 102′ and the main body 104′. A lid switch 110 can be connected to the main body 104′ and/or the lid 102′ to detect an opening or closing of the lid 102′, in some embodiments. In an example, the lid switch 110 can complete a circuit in response to the lid 102′ being opened and/or closed, which can be used to indicate the opening and/or closing of the lid 102′.

The pack 100′ can include indication lights 106A′, 106B, which can provide notifications to a user and/or be used to create a visual appeal to the pack. The pack 100′ can further include a switch 112, which can be used to operate various functions of the pack. For example, the switch 112 can be used to turn various features of the pack on or off. For instance, operating the switch 112 can tum a social networking function of the pack on or off and/or can turn a charging function of the pack on or off, although examples are not so limited.

FIG. 1C is an isometric bottom and side view of the pack 100′ depicted in FIG. 1B. The pack 100′ can include the lid 102′ and the body 104′. In addition, the pack 100′ can include the indication light 106A′, as well as the switch 112. In some embodiments, the pack 100′ can include a connection port 114. As shown in FIG. 1C, the connection port 114 can be a wired connection and include a data connection and/or a power connection. For example, the wired connection can include a USB connection and/or other type of wired data/electrical connections. In some embodiments, data can be transferred through the connection port 114 and/or power can be transferred through the connection port 114 to provide power to the electronic smoking article (e.g., pack and/or electronic cigarette) to charge a battery associated with the electronic smoking article, for example. The connection port 114 can also be a wireless connection that allows for transfer of data and/or power to the electronic smoking article. For example, the connection port 114 can include a wireless antenna and/or can be configured for non-contact inductive recharging such that the power may be transferred to the pack 100′ and it may be charged without being physically connected to an external power source.

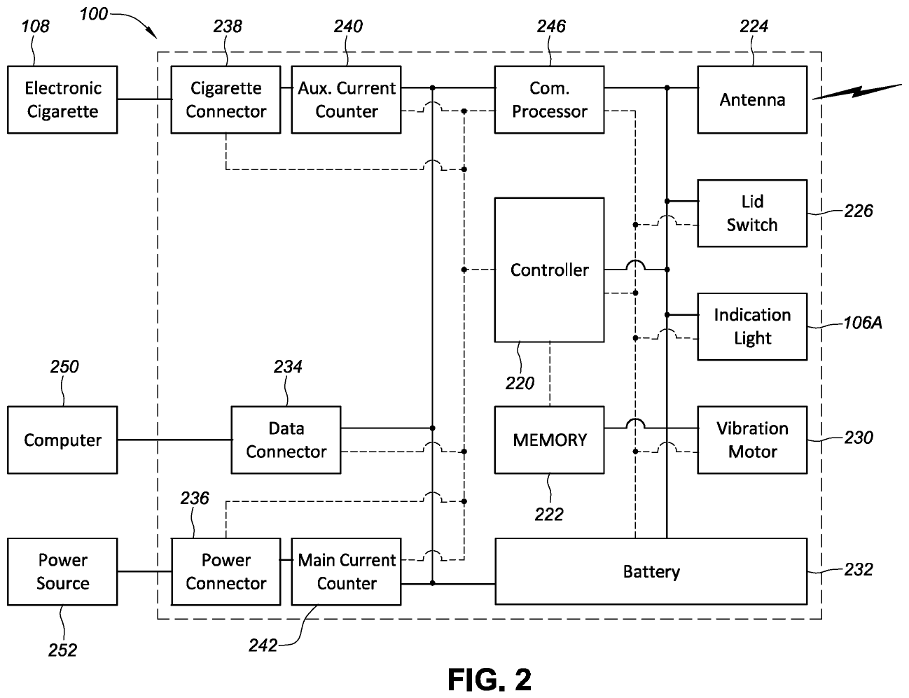

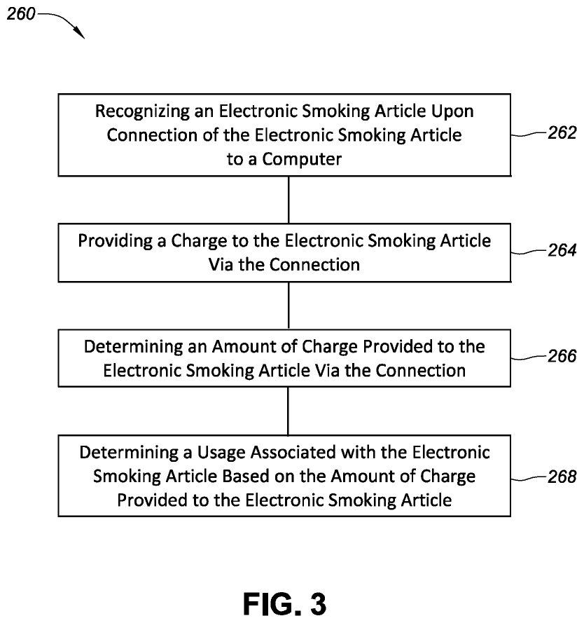

FIG. 2 is a schematic overview of a system incorporating a pack for electronic cigarettes (e.g., the pack 100 shown in FIG. 1), an electronic cigarette, a computer, and an external power source, in accordance with embodiments of the present disclosure. The pack 100 can include a computing device. For example, the computing device can include a controller 220 (e.g., processor) and a memory 222 coupled to the controller 220. Memory 222 can be in communication with the controller 220, which can be used to perform the method as discussed in relation to FIG. 3. The pack 100 can include an antenna 224. The antenna 224 can include a Wi-Fi module and/or a cellular module, enabling a wireless connection to a network, a computer 250, and/or another electronic smoking article. A communication processor 246 may carry out wired and/or wireless communications via the data connector 234 and/or the antenna 224. In addition, the communication processor 246 may carry out wired and/or wireless communications via the cigarette connector 238.

The pack 100 can include a lid switch 226, which can detect an opening or closing of the lid 102, in some embodiments. The pack 100 can include an indication light 106A, which can be used to provide an indication to a user of the pack 100. In addition, in some embodiments, the pack 100 can include a vibration motor 230, which can provide a vibration in response to an event occurring. For example, the event can include the pack 100 coming within a particular range of another pack, an indication of a particular power level associated with a battery 232 included in the pack, and/or another event.

In some embodiments, the data connector 234 can be a wired connection that is configured to provide a physical connection between a computer 250 and the pack 100 and/or the pack and another pack via a USB connection, for example, although other types of connections can be used (e.g., FireWire). In some embodiments, the data connector 234 can be a wireless connection, which can provide a wireless connection between the computer 250 and the pack 100 and/or between the pack 100 and another pack. In an example, the antenna 224 can provide for the wireless connection when the data connector 234 is wireless and/or includes a wireless module.

The pack 100 can include a power connector 236, which can be a wired connection (e.g., USB, FireWire) and/or a wireless connection. The wireless connection can provide a power connection via inductive charging in some embodiments, for example. In some embodiments, the data connector 234 and the power connector 236 can be provided in one connector. For example, a USB cable can be connected to a single connection, which includes a data connection and a power connection.

The cigarette connector 238 can be configured to connect to an electronic cigarette 108. For example, the electronic cigarette 108 and the cigarette connector 238 can have complementary terminals that are configured to connect with one another. In an example, the connection between the electronic cigarette 108 and the cigarette connector 238 can provide for a power connection and/or a data connection. Data can be exchanged via the pack 100 and a component of the electronic cigarette 108. For example, data can be exchanged via the pack 100 and a sensor, circuit, and/or computing device included in the electronic cigarette 108. In some embodiments, the cigarette connector 238 can be connected to the electronic cigarette 108 by a threaded-type connection, a non-threaded-type connection, a stationary connection, a push-in (pressing) connection, and/or the like.

In some embodiments, the battery 232 can provide power to the various components of the pack 100. With reference to FIG. 2, power connections between the various components is indicated by the solid lines, and communication connections between the various components is indicated by dotted lines. In an example, the battery 232 can be connected via a power connection directly and/or indirectly to the power connector 236, current counters 240, 242 (discussed further below), cigarette connector 238, data connector 234, communication processor 246, controller 220, memory 222, antenna 224, lid switch 226, indication light 106A, and/or vibration motor 230. In some embodiments, the pack 100 can include additional components (e.g., global positioning sensor) to which power may be provided by the battery 232. In an example, the controller 220 can be connected via a communication connection directly and/or indirectly to the cigarette connector 238, current counters 240, 242, data connector 234, power connector 236, communication processor 246, memory 222, antenna 224, lid switch 226, indication light 106A, vibration motor 239, and/or battery 232.

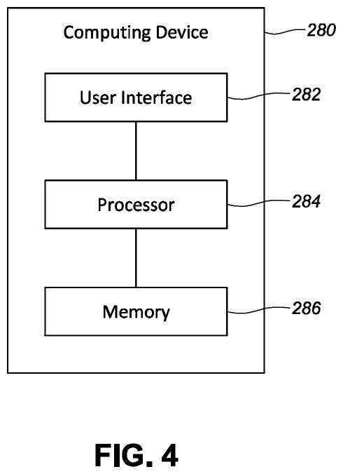

In some embodiments of the present disclosure, a connection can be created between an electronic smoking article and a computer 250. The computer 250 can include a processor and memory, such as the computing device 280 schematically shown in FIG. 4. For example, a connection can be created between the pack 100 and the computer 250. The connection can be a wired (e.g., physical) connection and/or a wireless connection. In some embodiments, data can be transferred between the pack 100 and the computer 250. In some embodiments, power can be transferred between the pack 100 and the computer 250. For instance, upon connection of the pack 100 to the computer 250, power can be transferred to the pack 100 to charge the battery 232.

Upon connection with the electronic smoking article, data can be transferred between the computer 250 and the electronic smoking article via the data connector 234. An electronic smoking article (e.g., pack 100, electronic cigarette 108) can include instructions that provide for control over different components in the electronic smoking article. Based upon the transferred data, a diagnostic associated with the electronic smoking article can be determined. In some embodiments, the diagnostic can be a usage characteristic associated with the electronic smoking article. The usage characteristic can include, for example, a charge provided to the electronic smoking article through an external power source 252 and/or an amount that the electronic smoking article is used, which can be determined from data that includes an amount of current transferred to the electronic smoking article. Additional information can be determined from the current transferred to the electronic smoking article, as further discussed below. In some embodiments, the diagnostic can be a specification associated with the electronic smoking article. For example, the diagnostic can include a specification such as a version of instructions stored on the electronic smoking article, which can be determined from data transferred from the electronic smoking article. For instance, data associated with the instructions and/or a version of the instructions can be stored on the memory of the electronic smoking article (e.g., memory 222 of the pack 100 and/or a memory of the electronic cigarette 108).

Upon connection of the computer 250 with the electronic smoking article, data can be transferred between the computer 250 and the electronic smoking article via the data connector 234. In some embodiments, the transferred data can include data that is indicative of and/or data that can be analyzed to determine the version of instructions that are stored on the memory of the electronic smoking article. In some embodiments, the electronic smoking article can initially be loaded with a version of instructions that can provide for control over different components of the electronic smoking article. However, in some instances, an error can exist in the instructions and/or a new version of one or more instruction sets can be created that provides for more effective and/or more efficient control over the different components in and/or aspects of using the electronic smoking article. Accordingly, it may be beneficial to know what version of instructions are currently loaded on the memory of the electronic smoking article. For instance, if an old version of instructions is loaded on the memory, it may be desirable to update the old version of instructions with a more current version of instructions.

A different version of instructions can be transferred to the electronic smoking article in response to a determination of the version of instructions stored on the electronic smoking article. For instance, based upon the version of instructions currently stored on the electronic smoking article, a determination can be made whether that version is a non-current version of instructions and/or if an error exists in that version of instructions. When a determination is made that the version of instructions currently stored on the electronic smoking article is a non-current version of instructions, and/or that an error exists in that version of instructions, a new or different version of instructions can be transferred to the electronic smoking article. For instance, if the version of instructions currently stored on the electronic smoking article is a non-current version, a new version of instructions and/or additional instructions may be transferred to the electronic smoking article and loaded on the electronic smoking article (e.g., on the memory 222 shown in FIG. 2). If the version of instructions currently stored on the electronic smoking article contains an error, a new or updated version of instructions and/or additional instructions may be transferred to the electronic smoking article to fix the error in the currently-stored instructions.

In some embodiments, the transferred data can include errors that have occurred with respect to instructions that are currently loaded on the electronic smoking article. For example, the electronic smoking article and/or computer 250 may include an event manager that includes an error logger that logs errors that have occurred with respect to instructions and/or hardware associated with the electronic smoking article. For instance, if a particular version of instructions loaded on the electronic smoking article has produced an error, the error can be recorded and stored in a memory (e.g., the memory of the electronic smoking article). Alternatively, errors can include a failure of a battery associated with the electronic smoking article to charge and/or an increased and/or decreased voltage and/or current produced by the battery associated with the electronic smoking article.

As discussed herein, in some embodiments, the characteristic can be a usage characteristic associated with the electronic smoking article. The usage characteristic can include, for example, a charge provided to the electronic smoking article via a power source, such as the external power source 252 schematically depicted in FIG. 2. Additional information can be determined from the usage characteristic, as further discussed herein.

In some embodiments, the diagnostic can be associated with a current delivered to a battery associated with the electronic smoking article. For instance, the diagnostic can be associated with a current delivered to the battery associated with the electronic smoking article (e.g., battery 232 associated with the pack or a battery associated with the electronic cigarette 108) to charge the battery. In some embodiments of the present disclosure, the charge delivered to the battery can be measured. For example, the electronic smoking article can include one or more current counters. For instance, the electronic smoking article can include a main current counter 242 and/or an auxiliary current counter 240 (both of which are schematically depicted in FIG. 2), although fewer current counters or more current counters can be employed in the electronic smoking article. In some embodiments, the electronic cigarette 108 can include a current counter configured to measure a current delivered to the battery (not shown) associated with the electronic cigarette 108.

In some embodiments, a main current counter 242 can be disposed between the power connector 236 and the battery 232 of the pack 100. Thus, as power is transferred from, for example, the external power source 252 to the battery 232, the main current counter 242 can determine how much current passes from the external power source 252 to the battery 232. In some embodiments, an auxiliary current counter 240 can be disposed between the cigarette connector 238 and the battery 232. Thus, as power is transferred from the battery 232 to the electronic cigarette 108 through the cigarette connector 238, the auxiliary current counter 240 can determine how much current passes from the battery 232 to the electronic cigarette 108. The determination of how much current passes from the external power source 252 to the battery 232 and/or from the battery 232 to the electronic cigarette 108 can be used to find a state of charge of the battery 232 in the pack and/or a battery in the electronic cigarette 108. The state of charge of the battery can be defined as an expression of a present capacity as a percentage of maximum capacity of the battery.

The state of charge of the battery 232 can be measured using various methods. For example, a voltage of the battery can be measured and used as a basis for calculating the state of charge of the battery. As a battery discharges, a voltage provided by the battery can change or vary. The voltage produced by the battery can change depending on how much of a charge is present in the battery. For example, as an amount of available charge in the battery decreases, the voltage produced by the battery can also decrease. However, problems can be associated with using the voltage of the battery for calculating the state of charge. For example, different cell types (e.g., lead acid, lithium-ion) can have dissimilar compositions that deliver varied voltage profiles. In addition, factors such as temperature can also have an impact on the voltage produced by the battery. As a temperature of the battery increases, for example, a voltage produced by the battery can increase. In contrast, as a temperature of the battery decreases, the voltage produced by the battery can decrease. As a result, measuring the state of charge of the battery using the voltage of the battery can lead to inconsistent and/or inaccurate determinations of the state of charge of the battery.

The state of charge of the battery can also be measured through determination of a specific gravity of a solution within the battery. For example, as a lead acid battery discharges an electrolyte in the battery, an acid (e.g., sulfuric acid) contained in an electrolyte solution of acid and water is consumed, and a concentration of the acid in the electrolyte solution is reduced. This in turn reduces a specific gravity of the electrolyte solution in direct proportion to the state of charge. Thus, through measurement of the specific gravity of the electrolyte solution, which can be performed through a hydrometer and/or an electric sensor, the state of charge of the battery can be determined. Changes in temperature can alter determinations of specific gravity, leading to errors in the determination of the state of charge of the battery. In addition, the electrolyte solution must be allowed to stabilize after charge and discharge before a specific gravity reading can be taken. Further, determining the state of charge of a battery through determinations of specific gravity is not normally suitable for cell chemistries other than lead acid batteries.

In some embodiments of the present disclosure, the state of charge of the battery can be determined using coulomb counting Coulomb counting involves integrating current over time of the current, which delivered a charge to the battery, to estimate a total transferred charge to the battery. A remaining capacity in a cell can be calculated by measuring the current entering (charging) and/or leaving (discharging) cells of the battery and integrating this over time. In an example, a charge transferred to the battery can be determined by coulomb counting using the following model:

Q=ΣI*Δt

where Q is the total charge transferred to the battery, I is the current transferred into and/or out of the battery, and Δt is a change in time over which the current is transferred into and/or out of the battery. In some embodiments of the present disclosure, the state of charge of the battery can be determined using joule counting, which involves integrating power over time to estimate a total transferred energy to the battery. In an example, a total transferred energy to the battery can be determined by joule counting using the following model:

E=ΣI*V*Δt

where E is the total energy transferred to the battery, I is the current transferred into and/or out of the battery, V is a voltage measured at a terminal of the battery, and Δt is a change in time over which the current is transferred into and/or out of the battery.

In some embodiments, the electronic smoking article can include a current counter disposed in the electronic smoking article, as discussed herein. In an example, the current counter can be a coulomb counter that can be configured to determine the current delivered to the battery associated with the electronic smoking article. A time over which the current is delivered to the battery associated with the electronic smoking article can be determine by the current counter, determined by the controller 220 of the electronic smoking article (e.g., through instructions executed by the controller), and/or determined by a processor associated with the computer 250. For example, when the electronic smoking article is being charged through the computer 250 (i.e., when the electronic smoking article is connected to the computer 250), the time that the electronic smoking article is charging can be recorded by the computer 250, the controller 220, and/or the current counters 240, 242. When the electronic smoking article is being charged via another power source such as external power source 252 (i.e., when the electronic smoking article is not connected to the computer 250 or when the connection to the computer does not deliver power to the electronic smoking article), the time that the electronic smoking article is charging can be recorded by the current counters 240, 242 and/or the controller 220. In some embodiments, a total charge transferred to the battery and/or the total energy transferred to the battery can be determined by the computer 250 (when the electronic smoking article is in communication with the computer 250), by each current counter 240, 242, and/or the controller 220.

As discussed herein, the “electronic smoking article” can include the electronic cigarette 108 and/or the pack 100. Accordingly, a battery associated with the electronic smoking article can include the battery (not shown in the figures) associated with the electronic cigarette 108 and/or the battery 232 associated with the pack 100. In some embodiments, a total amount of current delivered to the battery associated with the electronic cigarette 108 and/or a total amount of energy delivered to the battery associated with the electronic cigarette 108 can be determined via the auxiliary current counter 240. In some embodiments, a total amount of current delivered to the battery 232 associated with the pack 100 and/or a total amount of energy delivered to the battery 232 associated with the pack 100 can be determined via the auxiliary current counter 240.

In some embodiments, the battery 232 can be charged via the external power source 252, as discussed herein. The battery 232 can provide power to the various components of the pack 100. In addition, the battery 232 can be used to provide a charge to the battery of the electronic cigarette 108. For example, as shown in FIG. 1B, a plurality of electronic cigarettes can be inserted into the pack 100. The electronic cigarettes can connect with the pack via one or more cigarette connectors 238 (FIG. 2). In an example, the pack 100 can include one or more auxiliary current counters 240. For instance, in some embodiments, one auxiliary current counter can determine a total current delivered to a plurality of electronic cigarettes 108 and/or a total current delivered to each of the plurality of electronic cigarettes 108. Alternatively, the pack can include a plurality of auxiliary current counters, such that each of the plurality of auxiliary current counters is configured to measure a total current delivered to one or each of the plurality of electronic cigarettes 108.

In some embodiments, data can be exchanged via the pack 100 and a sensor, circuit, and/or computing device included in the electronic cigarette 108. Alternatively, and/or in addition, the electronic cigarettes 108 can accept a charge from the pack 100 via the cigarette connector 238. In an example, the charge provided to each individual electronic cigarette 108 can be monitored via one or more auxiliary current counters 240, as discussed herein. As the electronic cigarettes 108 are used (e.g., smoked), a charge of a battery that provides power to each of the electronic cigarettes 108 can be depleted.

In some embodiments, the battery 232 in the pack 100 can provide power to the battery of each electronic cigarette 108 in order to provide a charge to the battery of each electronic cigarette 108. As such, upon connection of the electronic cigarette 108 to the cigarette connector 238, the charge in the battery 232 in the pack 100 can be depleted in order to charge the battery in the electronic cigarette 108. In some embodiments, a substantially equivalent amount of energy may be transferred from the battery 232 in the pack to the battery in the electronic cigarette 108. As a result, the amount of charge in the battery in the electronic cigarette 108 and the amount of charge in the battery 232 in the pack 100 can correspond to a usage of the electronic cigarette 108 by the user.

In some embodiments of the present disclosure, an amount of, for example, juice in the electronic cigarette 108 can be determined based upon the charge of the battery associated with at least one of the battery of the individual electronic cigarette 108 and the charge of the battery 232 of the pack 100. In an example, a particular amount of charge in the battery of the electronic cigarette 108 and/or the battery 232 of the pack 100 can provide for a particular vape or smoke time associated with the electronic cigarette 108. For example, an electronic cigarette 108 with a charged 1000 milliampere hour (mAh) battery may be able to provide for a continuous use of an electronic cigarette 108 for a particular time (e.g., 30 minutes).

In some embodiments, a juice usage rate associated with the electronic cigarette 108 can be determined. For example, an electronic cigarette 108 that holds 10 milliliters of juice can provide for 3000 puffs that each averages 2.5 seconds long. As such, the electronic cigarette 108 can provide approximately 7500 seconds of total use with 10 milliliters of juice, which results in a usage rate of juice of 0.0013 milliliters per second. As discussed herein, in an example, a charged battery in the electronic cigarette 108 can provide for a continuous use of the electronic cigarette 108 for a particular time. In some embodiments, an amount of charge in the battery can be correlated with an amount of juice used by the electronic cigarette 108.

For example, the amount of charge in the battery of the electronic cigarette 108 can be determined from data obtained from the auxiliary current counter 240, and/or a current counter included in the electronic cigarette 108. For instance, as a charge is provided to the battery in the electronic cigarette 108, an amount of current and/or power transferred to the battery can be determined, as discussed herein. Accordingly the amount of current and/or power transferred to the battery of the electronic cigarette 108 can be correlated with an amount of juice used by the electronic cigarette 108. For instance, if a total current required to charge the battery is approximately 3500 coulombs, the total current can be correlated with 30 minutes of continuous use of the electronic cigarette 108 (e.g., 1800 seconds). An amount of juice in the electronic cigarette 108 can be correlated with the amount of use of the electronic cigarette 108. For example, if a determination is made that the electronic cigarette 108 was used for 1800 seconds, based upon the total current transferred to the battery, the device can have approximately 7500 seconds minus 1800 seconds of use left, which would be 5700 seconds of use left before the juice in the electronic cigarette 108 runs out. In addition, a total amount of juice used by the device can be determined, which can be calculated by dividing an amount of smoking time that the device was used by the total amount of smoking time available from the device. For example, 1800 seconds divided by 7500 seconds equals 0.24, which would be a fractional amount of juice that was used in the 1800 seconds. As such, 0.24 times the total amount of juice available would equal an amount of juice that was used, which would be 0.24 times 10 milliliters, which would equal 2.4 milliliters of juice used, meaning that 7.6 milliliters of juice remains in the electronic cigarette 108. Accordingly, the amount of juice used can be correlated with the amount of coulombs transferred to the battery of the electronic cigarette 108. For example, 2.4 milliliters of juice were used per 3500 coulombs of charge transferred to the battery of the electronic cigarette 108.

In some embodiments, a number of puffs of the electronic cigarette 108 can be determined based upon the charge of the battery associated with the electronic cigarette 108. For example, as discussed herein, if a total current required to charge the battery is approximately 3500 coulombs, the total current can be correlated with 30 minutes of continuous use of the electronic cigarette 108 (e.g., 1800 seconds), which would equate to 720 2.5-second-long puffs. As such, based upon the current transferred to the battery, a usage of the electronic cigarette 108 can be determined. In some embodiments, each coulomb transferred to the battery of the electronic cigarette 108 can be correlated with a smoking time. For example, in the present example, if 3500 coulombs provides 1800 seconds of smoking time, a determination of smoking time per coulomb transferred can be made. For instance, each coulomb transferred would provide 0.51 seconds of smoking time.

In some embodiments, a determination of a time associated with a puff of the electronic cigarette 108 can be made. For example, bench top tests can be made to determine how long an average puff lasts. Alternatively, in some embodiments of the present disclosure, information can be obtained from the electronic cigarette 108 regarding how long each puff of the electronic cigarette 108 lasts. In some embodiments, the electronic cigarette 108 can provide an average time associated with how long each puff lasts to the controller 220 via a data connection provided by the cigarette connector 238. Alternatively, the electronic cigarette 108 can provide data associated with each puff to the controller 220, for which the controller can determine an average. In an example, the time of each puff can be determined through a sensor included in the electronic cigarette 108. For instance, the sensor can include an air flow sensor and/or a microphone, which is activated when the user draws air through the electronic cigarette 108. Instructions can be executed by a controller in the electronic cigarette 108 and/or the controller 220 in the pack 100 to transfer the data from the sensor included in the electronic cigarette 108 to the pack 100. In some embodiments, the data can then be transferred to the computer 250 for determination of an average puff time.

In some embodiments, when a plurality of electronic cigarettes 108 are included in the pack 100, each electronic cigarette 108 can include an identifier, such that data associated with each electronic cigarette 108 can be gathered. For example, an amount that each electronic cigarette 108 is used can be tracked individually. For instance, a time that each electronic cigarette 108 is used can be tracked (e.g., total smoking time), or an amount of juice left in each electronic cigarette 108, as well as other data associated with each electronic cigarette 108, can be tracked and/or recorded, as discussed herein. In addition, a number of cycles associated with each battery can be determined. For instance, a number of times that each electronic cigarette 108 has been discharged and recharged can be determined.

In some embodiments of the present disclosure, a usage behavior of a user of the electronic smoking article can be determined based upon the current transferred to the battery associated with the electronic smoking article, as discussed herein. For example, the controller can record a time and/or an amount of current transferred to the battery associated with the electronic smoking article. In an example, when a user uses the electronic cigarette 108, they can remove the electronic cigarette 108 from the pack 100 to smoke the electronic cigarette 108. When finished with the electronic cigarette 108, the user can place the electronic cigarette 108 in the pack 100 for storage and/or to receive a charge from the pack battery 232. In some embodiments, a time can be recorded by the controller 220 to determine a time between when the user removes the electronic cigarette 108 from the pack 100 and places the electronic cigarette 108 back into the pack 100. In some embodiments, an amount which the electronic cigarette 108 has been used can also be determine, as discussed herein, by determining a current transferred to the electronic cigarette 108 with the auxiliary current counter. As such, habits of the user can be determined and stored in some examples.

In some embodiments, the amount of current transferred to the battery 232 associated with the pack 100 can be used to determine a total amount of juice that has been used by electronic cigarettes 108 stored in the pack 100, a smoke time associated with the electronic cigarettes 108 stored in the pack 100, a number of puffs associated with the electronic cigarettes 108 stored in the pack 100, a juice usage rate associated with the electronic cigarettes 108 stored in the pack 100, as well as other usage statistics associated with the electronic cigarettes, as discussed herein. The battery 232 can provide a charge to the batteries associated with the electronic cigarettes. As such, the current transferred from the battery 232 to the batteries associated with the electronic cigarettes 108 can be representative of an amount of power that has been used by the electronic cigarettes 108, as a result of being smoked. The current transferred from the battery 232 to the batteries associated with the electronic cigarettes 108 can be determined via the main current counter 242 and/or the auxiliary current counter 240.

In some embodiments, upon connection of the pack 100 to the computer 250 via the data connector 234, the data associated with the electronic cigarette 108 and/or the pack 100 can be transferred to the computer 250. For example, an amount that the electronic cigarette 108 is used (e.g., total smoking time), an amount of juice remaining in the electronic cigarette 108, a number of charge and/or discharge cycles associated with the battery in each electronic cigarette 108 and/or the pack 100, etc. can be transferred to the computer 250. In some embodiments, a life of the battery in each electronic cigarette 108 and/or the pack 100 can be tracked based upon the number of charge and/or discharge cycles. For example, a determination of when the battery in an electronic cigarette or in a pack should be replaced can be made based upon the number of charge and/or discharge cycles.

In some embodiments of the present disclosure, a credit can be offered to a user in response to a transfer of data occurring between the computer 250 and the electronic smoking article. In an example, the credit may be offered to the user in response to the transfer of data occurring between the computer 250 and the electronic smoking article. Alternatively, the credit may be offered to the user in response to a determination of a diagnostic associated with the electronic smoking article. In an example, if the diagnostic is unable to be determined from the data, in some embodiments, the credit may not be offered to the user. The credits can include offers and/or coupons for electronic smoking articles and related products. In some cases the credits can be points, which can be provided to the user in response to the transfer of data. As such, the individual can be prompted to continually transfer data regarding the electronic cigarette 108 and/or the pack 100 to the computer 250. In some embodiments, the credits can be stored on a memory of the device. Alternatively, and/or in addition, the credits can be stored on the computer 250. In some embodiments, as a user accumulates credits, the credits can be sent in an email, text message, and/or a physical credit can be mailed to the user.

In an example, data associated with a use of the electronic cigarette 108 and/or a use of the pack 100 can be transferred to the computer 250. The data associated with the use of the electronic cigarette 108 and/or the use of the pack 100 can be used by a maker of the electronic cigarette 108 and/or by a third party in research, product development, and/or marketing In an example, based upon use of the electronic cigarette 108, offers and/or coupons can be generated that are tailored to the user. If the user uses the electronic cigarette 108 during a particular time each day, a coupon for a cartomizer loaded with a particular flavor of juice can be offered to the user. For instance, if the user uses the electronic cigarette 108 every morning, a coupon can be offered to the user for a particular flavor, such as hazelnut.

In some embodiments, the transferred data can include data that is indicative of, and/or data that can be analyzed to determine, the version of instructions that are stored on the memory of the electronic smoking article (e.g., memory 222 of the pack 100, memory of the electronic cigarette 108), as discussed herein. In addition, the transferred data can include data associated with instructions that are being transferred to the electronic smoking article. Alternatively, transferred data can include error data, such as a failure of a battery associated with the electronic smoking article to charge and/or an increased and/or decreased voltage and/or current produced by the battery associated with the electronic smoking article.

In some embodiments, a first amount and/or type of credit (e.g., credits to be applied towards different types of products) may be provided to a user for sharing usage data associated with that particular user's use of the electronic cigarette 108; and a second, different amount and/or type of credit may be provided to that same user for sharing data associated with the specific instruction set or sets onboard the electronic cigarettes or packs used by the user and/or related error data. For instance, a greater amount of credits (and/or a lesser amount of credits) may be provided to the user for sharing data associated with instructions and/or error data than for sharing data associated with usage of the electronic cigarette 108.

FIG. 3 is a method flow diagram that depicts a method 260 for determining a diagnostic associated with an electronic smoking article. In an example, determining the diagnostic associated with the electronic smoking article can include establishing a connection between the electronic smoking article and a computer. In some embodiments, determining the diagnostic associated with the electronic smoking article can include transferring data between the electronic smoking article and the computer. The method 260 can include recognizing 262 an electronic smoking article upon connection of the electronic smoking article to the computer. In some examples, the electronic smoking article can be a pack that is configured for storing one or more of a plurality of individual electronic cigarettes; and the computer can be a personal computer, smart phone, tablet, etc. In some examples, the electronic smoking article can be an electronic cigarette. In an example, the pack can be connected with the computer via a wired and/or wireless connection. Upon connection of the pack to the external computer 250 (FIG. 2), a computer in the pack (e.g., the controller 220 and the memory 222), as discussed herein, and/or the external computer 250 can recognize one another. In some embodiments, the electronic smoking article can include each individual electronic cigarette in the pack. In some embodiments, as discussed herein, each individual electronic cigarette can include an identifier that can be recognized by the computer 250. In some embodiments, the electronic cigarettes 108 can each include a computer (e.g., processor and memory), which can recognize the computer 250 and/or can be recognized by the computer 250, upon connection of the electronic cigarette 108 to the computer. For example, the electronic cigarettes 108 can be directly connected to the computer 250 and can recognize and/or be recognized by the computer 250 and/or the electronic cigarettes 108 can be indirectly connected to the computer 250 via the cigarette connector 238 (e.g., pack 100) and can recognize and/or be recognized by the computer 250. In some embodiments, each individual electronic cigarette can include an identifier, as discussed herein, which can be recognized by the computer.

In some embodiments, the method 260 can include a step 264 of providing a charge to the electronic smoking article via the connection between the electronic smoking article and the computer. In an example, a battery of the pack and/or electronic cigarette can be charged via the connection. The method 260 can include a step 266 of determining an amount of charge and/or power provided to the electronic smoking article via the connection, in some embodiments. For example, as discussed herein, the electronic smoking article can include one or more current counters. For instance, the electronic smoking article can include a main current counter 242 and/or an auxiliary current counter 240, although fewer current counters or more current counters can be employed in the electronic smoking article. The main current counter 242 can determine an amount of charge provided to the battery 232 of the pack and the auxiliary current counter 240 can determine an amount of charge provided to the electronic cigarette, in an example.

In some embodiments, the method 260 can include a step 268 of determining a usage associated with the electronic smoking article based upon the amount of charge provided to the electronic smoking article. In some embodiments, a determination of the usage can be based upon a determination of how much current is provided to the battery of the electronic smoking article, which can be provided via the current counter, as discussed herein. As also discussed herein, as the electronic smoking article is used, the battery can be depleted by a particular amount. Thus, depletion of the battery can be correlated with the usage of the electronic smoking article. As such, in an example, the amount of charge provided to the battery of the electronic smoking article to recharge the battery can be correlated with a usage of the electronic smoking article. As discussed herein, an amount of juice used by and/or remaining in the electronic cigarette can be determined, along with other information such as a usage behavior of a user, for example.

In some embodiments, the method 260 can include determining the usage associated with the electronic smoking article based upon an amount of charge that has previously been provided to the electronic smoking article. For example, as discussed herein, as the electronic smoking article is used, a charge in the battery associated with the electronic smoking article can be depleted. The electronic smoking article can be connected to a power source to recharge the battery associated with the electronic smoking article. In some embodiments, the amount of charge that has previously been provided to the electronic smoking article can be summed, which can be equivalent to a total usage of the electronic cigarette.

In some embodiments, the method 260 can include determining an amount of juice stored in the electronic smoking article, based upon the amount of charge and/or current that has been provided to the electronic smoking article. In an example, as the electronic smoking article is used, a charge of the battery can be depleted. As such, the amount of charge and/or current provided to a battery of the electronic smoking article to recharge the battery can be correlated with the usage of the electronic smoking article. In some embodiments, the electronic smoking article can be an electronic cigarette and the usage of the electronic cigarette can be determined based upon the amount of charge and/or current provided to the electronic cigarette. For example, an amount of juice used by the electronic cigarette can be determined. The amount of juice stored in the electronic cigarette (e.g., the amount of remaining juice stored in the electronic cigarette) can thus be determined.

FIG. 4 is a diagram of a computing device for determining a diagnostic associated with the electronic smoking article. Computing device 280 can be used to perform the method as discussed in relation to FIG. 3. As shown in FIG. 4, computing device 280 can include a user interface 282. User interface 282 can be a graphic user interface (GUI) that can provide (e.g., display and/or present) information to, and/or receive information (e.g., data and/or images) from, a user (e.g., operator) of computing device 280. For example, user interface 282 can include a screen that can provide information to a user of computing device 280 and/or receive information entered via the screen (e.g., touch screen) by the user. However, embodiments of the present disclosure are not limited to a particular type of user interface 282.

As shown in FIG. 4, computing device 280 can include a processor 284 (e.g., controller) and a memory 286 coupled to the processor 284. Memory 286 can be any type of storage medium that can be accessed by the processor 284 to perform various examples of the present disclosure. For example, memory 286 can be a non-transitory computer readable medium having computer readable instructions (e.g., computer program instructions) stored thereon that are executable by the processor 284 to determine the diagnostic associated with the electronic smoking article, according to one or more embodiments of the present disclosure.

Memory 286 can be volatile or nonvolatile memory. Memory 286 can also be removable (e.g., portable) memory, or non-removable (e.g., internal) memory. For example, memory 286 can be random access memory (RAM) (e.g., dynamic random access memory (DRAM) and/or phase change random access memory (PCRAM)), read-only memory (ROM) (e.g., electrically erasable programmable read-only memory (EEPROM) and/or compact-disk read-only memory (CD-ROM)), flash memory, a laser disk, a digital versatile disk (DVD) or other optical disk storage, and/or a magnetic medium such as magnetic cassettes, tapes, or disks, among other types of memory.

Further, although memory 286 is illustrated as being located in computing device 280, embodiments of the present disclosure are not so limited. For example, memory 286 can also be located internal to another computing resource (e.g., enabling computer readable instructions to be downloaded over the Internet or another wired or wireless connection). Processor 284 can execute the executable instructions stored in memory 286 to establish a data connection between an electronic smoking article and a computer upon connection of the electronic smoking article to the computer.

In an example, the electronic smoking article can include a pack configured to hold one or more of a plurality of individual electronic cigarettes. In an example, the computer, which can be for example a desktop computer, smartphone, tablet, etc., as discussed herein, can be connected with the electronic smoking article via a wired and/or a wireless connection. In some embodiments, the computer and/or the electronic smoking article can recognize one another upon wired or wireless connection with one another. Processor 284 can execute the executable instructions stored in memory 286 to transfer data to or from the electronic smoking article.

In some embodiments, data can be transferred from the electronic smoking article and received by the computer. In some embodiments, the data can include a diagnostic, as discussed herein. Processor 284 can execute the executable instructions stored in memory 286 to perform an operation on the transferred data to determine a diagnostic related to the electronic smoking article. In some embodiments, the diagnostic can be associated with an error associated with the electronic smoking article, as discussed herein. For example, the error can be an error in operation of the electronic smoking article.

In an example, the error can include a hardware error, instruction error, and/or another type of error that results in an unfavorable user experience. The error can include, for example, events such as a low battery associated with the electronic smoking article, a battery that is bad and will not charge and/or will only charge at a reduced rate or to a reduced amount, a broken sensor associated with the electronic smoking device, and/or an unexpected low level of juice associated with the electronic smoking article. In an example, an unfavorable user experience can be caused by an event that causes the electronic smoking article to not function as it was designed. For instance, the electronic smoking article operates at a reduced level (e.g., producing a lower-than-expected amount of vapor) and/or does not operate at all. In some embodiments, the event can be associated with instructions that are loaded onto a memory of the electronic smoking article that are out of date and/or instructions that include an error that is associated with the code implementing the instructions.

In some embodiments, processor 284 can execute the executable instructions stored in memory 286 to perform a mitigating action based upon the error in operation of the electronic smoking article. In some examples, the mitigating action can include notifying a user of the electronic smoking article of the error. For instance, upon establishment of the data connection between the electronic smoking article and the computer, instructions can be executed on the computing device to open an application on the computing device. In some embodiments, the application can display diagnostics associated with the electronic smoking article to the user. For instance, the application can display to the user a diagnostic associated with the pack and/or diagnostics associated with one or more of the plurality of electronic cigarettes.

In an example, the application can be a native application, a web based application, and/or a hybrid application. In an example, a native application is an application program that has been developed for use on a particular platform or device. The native application can be stored on a computing device and installed on the computing device. In some embodiments, data associated with the native application can be stored on the device, although data can be stored remotely and accessed by the native application. In some embodiments, the application can be a web application, which is an application program that is stored on a remote server and delivered over the Internet through a browser interface (e.g., of the computing device). In some embodiments, the application can be a hybrid application, which is an application that combines elements of native and web applications. In an example, hybrid applications can function whether the computing device is online or offline. In some embodiments, hybrid applications can be developed for a specific platform and installed on a computing device. Hybrid applications can be integrated into a computing device's file system and/or integrated with web-based services. In some embodiments, the hybrid application can include an embedded browser to improve access to dynamic online content.

In an example, the application can display diagnostics associated with the pack, such as a version of instructions that are loaded onto a memory of the pack, a level of charge associated with the battery in the pack, an indication of the chargeability of the battery in the pack, another indicator of the health of the battery in the pack, an amount of credits that have been provided to the user for transfer of data, any instruction and/or hardware errors associated with the pack, as well as other diagnostics discussed herein. The application can display diagnostics associated with one or more of the electronic cigarettes, such as a version of instructions that is loaded onto a memory of the electronic cigarette, a level of charge associated with each of the electronic cigarettes, an amount of juice in each of the electronic cigarettes, an indication of the chargeability of the battery in each of the electronic cigarettes, another indicator of the health of the battery in each of the electronic cigarettes, as well as other diagnostics discussed herein.

In some examples, the application can display a visual representation of the pack and of one or more of the electronic cigarettes in the pack. For instance, a top, side, and/or front view of the pack depicted in FIG. 1B can be displayed to the user. In addition, diagnostics associated with each electronic cigarette can be displayed to the user. By displaying the visual representation of the pack and of one or more of the electronic cigarettes in the pack, the user can identify each electronic cigarette and the diagnostics associated with each electronic cigarette. For instance, if an error has occurred with the battery in one of the electronic cigarettes, an error has occurred in a version of instructions loaded onto one of the electronic cigarettes, and/or a level of juice is low or has been depleted in one of the electronic cigarettes, for example, the user can identify that electronic cigarette from the visual representation; and the user can take a corrective action, such as discarding the electronic cigarette, refilling the electronic cigarette with juice, replacing a battery in the electronic cigarette, updating the instructions loaded on the electronic cigarette, etc. In some embodiments, the computing device 280 can provide recommendations on how to resolve the error, as discussed herein.

In some embodiments, the user can position a pointing device (e.g., arrow) over one of the electronic cigarettes and/or the pack, and a diagnostic associated with the electronic cigarette and/or pack can then be displayed in response to the positioning of the pointing device. In an example, the user can “click” on one of the electronic cigarettes and/or on the pack to display the diagnostic information. Alternatively, and/or in addition, the information can be automatically displayed upon connection of the pack and/or electronic cigarette with the computer and/or upon selection of a tab displayed on the application.

As a complexity of electronic smoking articles increases, a chance of a component (e.g., hardware and/or instructions) associated with the electronic smoking article malfunctioning can also increase. The application can be beneficial in this regard, because it can provide diagnostics associated with the electronic cigarettes and/or pack in a format that is easy for a user to understand and can allow for a user to determine if a problem exists with the electronic smoking article (e.g., a pack or electronic cigarette). In addition, the application can allow for a user to fix a problem that exists with the electronic smoking article. As such, the application can help a user diagnose possible problems without contacting a customer support department, which can save resources (e.g., time and/or money) of a provider of the electronic smoking article and/or a provider of customer support associated with the electronic smoking article; and which can save a user from the hassle and frustration of having to return the electronic smoking article to a vendor when only a relatively minor adjustment/action may be all that is necessary to restore/ensure full performance of the electronic smoking article.

In some embodiments of the present disclosure, the mitigating action can include notifying a user of the electronic smoking article of the error in operation of the electronic smoking article. In an example, upon connection of the electronic smoking article to the computer, the notification can be provided to the user. For instance, the notification can be provided on the user interface 282 of the computing device 280. In some embodiments, the notification can be provided automatically upon connection of the electronic smoking article to the computer. Alternatively, the notification can be provided to the user in response to the user making one or more selections via the user interface.

In some embodiments, the mitigating action can include recommending a corrective action to the user to correct the error based upon an analysis of the error. In an example, the corrective action can be customized to the error. For instance, if the error is associated with a bad battery in the electronic smoking article, the corrective action can include replacing the electronic smoking article and/or replacing the battery in the electronic smoking article. If the error is associated with low juice in the electronic cigarette, the corrective action can include filling the electronic cigarette with juice and/or replacing the electronic cigarette. If the error is associated with out of date instructions on the electronic smoking article, the corrective action can include updating the instructions associated with the electronic smoking article.

In some embodiments of the present disclosure, processor 284 can execute the executable instructions stored in memory 286 to contact a vendor and/or third party (e.g., service department) in response to determination of the diagnostic associated with the error in operation of the electronic smoking article. In an example, information associated with the error can be provided to the vendor and/or third party. In some embodiments, the information can be used for product development, marketing, trouble shooting of other electronic smoking articles, etc. In some embodiments, the vendor and/or third party can use the information associated with the error to contact the user regarding the error in order to provide assistance in resolving the error.

In some embodiments, a credit can be provided to the user, as discussed herein, in response to the error. For example, the credit can be provided to the user in response to an error in the instructions loaded onto the electronic smoking device; in response to an error that renders the device unusable (e.g., an error that prevents the battery from being charged, low level of juice, etc.), a credit can be offered to the user as a result of the user being inconvenienced by the error. In some embodiments, the credit can include a discount that can be applied towards a new electronic smoking article.

Embodiments are described herein of various devices, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment(s) is included in at least one embodiment. Thus, appearances of the phrases “in various embodiments,” “in some embodiments,” “in one embodiment,” or “in an embodiment,” or the like, in places throughout the specification, are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

Although at least one embodiment for determining a diagnostic associated with an electronic smoking article has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the devices. Joinder references (e.g., affixed, attached, coupled, connected, and the like) are to be construed broadly and can include intermediate members between a connection of elements and relative movement between elements. As such, joinder references do not necessarily infer that two elements are directly connected and in fixed relationship to each other. It is intended that all matter contained in the above description or shown in the accompanying drawings shall be interpreted as illustrative only and not limiting. Changes in detail or structure can be made without departing from the spirit of the disclosure as defined in the appended claims.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed:

1. A non-transitory computer-readable medium storing instructions executable by a computer to cause the computer to:
create a connection with an electronic smoking article;
transfer data between the computer and electronic smoking article, upon connection with the electronic smoking article; and
determine a diagnostic associated with the electronic smoking article based upon the transferred data.

2. The medium of claim 1, wherein the diagnostic includes a version of instructions stored on the electronic smoking article.

3. The medium of claim 2, comprising instructions to transfer a different version of instructions to the electronic smoking article in response to a determination of the version of instructions stored on the electronic smoking article.

4. The medium of claim 1, wherein the diagnostic is associated with a current delivered to a battery associated with the electronic smoking article.

5. The medium of claim 4, further comprising instructions executable to receive data from a coulomb counter disposed in the electronic smoking article, wherein the coulomb counter is configured to determine the current delivered to the battery associated with the electronic smoking article.

6. The medium of claim 5, wherein:
the electronic smoking article is an individual electronic cigarette; and
the battery associated with the electronic smoking article is a battery of the individual electronic cigarette.

7. The medium of claim 5, wherein the battery associated with the electronic smoking article is a battery of a pack configured for storing a plurality of individual electronic cigarettes.

8. The medium of claim 5, further comprising instructions executable to determine an amount of juice in the electronic cigarette based upon the current delivered to the battery of the pack configured for storing the plurality of individual electronic cigarettes.

9. The medium of claim 5, further comprising instructions executable to determine a usage behavior of a user of the electronic smoking article based upon the current delivered to the battery associated with the electronic smoking article.

10. The medium of claim 1, further comprising instructions executable to provide a credit to the user in response to the transfer of data between the computer and electronic smoking article.

11. The medium of claim 1, further comprising instructions executable to provide a credit to a user in response to the determination of the diagnostic associated with the electronic smoking article, wherein the credit provided in response to the determination of the diagnostic associated with the electronic smoking article is different than the credit provided in response to the transfer of data.

12. A system comprising a processing resource in communication with a non-transitory computer readable medium having instructions executed by the processing resource to:
establish a data connection between an electronic smoking article and a computer upon connection of the electronic smoking article to the computer;
receive data from the electronic smoking article;
perform an operation on the transferred data to determine a diagnostic related to the electronic smoking article, wherein the diagnostic is associated with an error in operation of the electronic smoking article; and
perform a mitigating action based upon the error in operation of the electronic smoking article.

13. The system of claim 12, wherein the connection of the electronic smoking article to the computer is at least one of a wired and a wireless connection.

14. The system of claim 12, wherein the mitigating action notifies a user of the electronic smoking article of the error.

15. The system of claim 14, wherein the mitigating action recommends a corrective action to the user to correct the error based upon an analysis of the error.

16. The system of claim 15, further comprising instructions executable by the processing resource to:
contact a service department in response to determination of the diagnostic associated with the error; and
provide information associated with the error to the service department.

17. A method, comprising:
recognizing an electronic smoking article upon connection of the electronic smoking article to a computer;
providing a charge to the electronic smoking article via the connection;
determining an amount of charge provided to the electronic smoking article via the connection; and
determining a usage associated with the electronic smoking article based upon the amount of charge provided to the electronic smoking article.

18. The method of claim 17, wherein the method includes determining the usage associated with the electronic smoking article based upon an amount of charge that has previously been provided to the electronic smoking article.

19. The method of claim 17, wherein the method includes determining an amount of juice stored in the electronic smoking article based upon the amount of charge provided to the electronic smoking article.

20. The method of claim 17, wherein the electronic smoking article is an electronic cigarette and the usage of the electronic cigarette is determined based upon the amount of charge provided to the electronic cigarette.
